(19) United States
(12) Patent Application Publication
(10) Pub. No.: US 2014/0099711 A1
(43) Pub. Date: Apr. 10, 2014

(54) METHODS AND SYSTEMS FOR OPTIMIZING PERFUSION CELL CULTURE SYSTEM

(71) Applicant: Bayer HealthCare LLC, Berkeley, CA (US)

(72) Inventors: Yuval Shimoni, Kensington, CA (US); Volker Moehrle, Cologne (DE); Venkatesh Srinivasan, Sunnyvale, CA (US)

(21) Appl. No.: 14/049,676

(22) Filed: Oct. 9, 2013

Related U.S. Application Data
(60) Provisional application No. 61/712,190, filed on Oct. 10, 2012.

(51) Int. Cl. C07K 14/755

(52) U.S. Cl. CPC C07K 14/755
USPC 435/354; 435/325; 435/352; 435/358; 435/369; 435/289.1

(57) ABSTRACT

Methods and perfusion culture systems are disclosed. The systems and methods relate to decreasing the starting perfusion rate, resulting in increased residence time of the cells in the bioreactor and the cell retention device, and/or concomitantly increasing the starting bioreactor volume or decreasing the starting cell retention device volume, or both. Other method embodiments include increasing the concentrations of individual components of the tissue culture fluid, and adding a stabilizer of the degradation of the recombinant protein.

### RELATED APPLICATIONS

The present application claims priority from U.S. Provisional Patent Application Ser. No. 61/712,190, filed Oct. 10, 2012, entitled “METHODS AND SYSTEMS FOR OPTIMIZING PERFUSION CELL CULTURE SYSTEM” (Attorney Docket No. BHC125019(BH-021L)), which is hereby incorporated herein by reference in its entirety for all purposes.

### BACKGROUND

Recombinant proteins, such as rhFVIII (recombinant human factor VIII protein, which is an active ingredient of Kogenate® FS, or KG-FS, produced by Bayer Healthcare, Berkeley, Calif.), are often produced in a perfusion continuous cell culture process. A key controlled parameter in this system is the cell specific perfusion rate (also referred to herein as perfusion rate or as CSPR), which can be calculated as volume of perfused medium per cell per day (volume/C/D) or in volumes per day. Cell culture medium contributes significantly to overall production cost and is one reason why efforts are placed in using as low a perfusion rate that is optimal with respect to cell health and/or product yield and product quality. Further, if protein yield could be maintained, a lower perfusion rate could increase plant capacity and provide flexibility in production with minimal changes to infrastructure.

A relatively high perfusion rate helps assure that sufficient nutrients are provided to the cell culture, but it also dilutes the product, resulting in larger harvest volumes. On the other hand, a low perfusion rate would reduce product dilution, but could impact its stability. For example, increased residence time of the molecule in the conditions in the bioreactor could result in the molecule being exposed to proteases or other factors that could promote its degradation. The lower perfusion rate could also impact cellular performance if a nutrient becomes limiting in its concentration (or if byproducts build-up). Thus, merely lowering the perfusion rate is not sufficient.

The lowest perfusion rate that would provide sufficient nutrients and byproduct clearance for optimum cellular production of the protein product would therefore result in higher yields while requiring less tissue culture medium (also referred to herein as tissue culture fluid, tissue/cell culture media, or medium/media)—as long as the change in perfusion rate does not impact product stability. Thus, the perfusion rate should be optimized for cellular specific productivity and for product stability.

Changes in perfusion rate also affect the residence time (the average time that the cells and the product are exposed to the system's unit-operational conditions). Two key unit operations of a perfusion bioreactor system for producing recombinant proteins, such as recombinant FVIII, take place in the bioreactor and the cell retention device (also referred to herein as CRD), e.g., a settler. The bioreactor is optimized and controlled for ideal cell culture conditions (e.g., physiological temperature and adequate oxygenation), while typical cell retention devices are designed and optimized to retain and recirculate cells back to the bioreactor. Since the CRD is not typically designed to provide the ideal cultivation conditions of the bioreactor, the combination of high cell concentration and non-ideal conditions may be in an undesirable state. To mitigate these conditions, strategies such as cooling are employed to lower the metabolic rate of the concentrated cell mass. Typically, the conditions in the cell retention device are expected to reduce cell metabolism, which in turn may reduce cellular productivity.

In a perfusion system, cells (and product/byproduct) are continuously cycling between the bioreactor and the cell retention device. Cells are thus cycling between conditions favoring cellular productivity (i.e., in the bioreactor) and conditions where productivity is generally lower (e.g., in the CRD). The problem of cells in a perfusion system spending significant time in an external suboptimal environment (e.g., within a CRD) is well recognized in the industry (See Bonham-Carter and Shevitz, BioProcess Intl. 9(9) October 2011, pp. 24-30). Moreover, the longer cells reside in the CRD may result in the recovery taking longer once the cells return to the bioreactor. This may result in a further reduction in system productivity.

Recombinant protein product, such as FVIII, can be harvested through continuous media collection. FVIII product activity also decreases over time at temperatures used in the bioreactor. Thus, increasing residence time by decreasing perfusion rate may result in lower accumulation of active recombinant protein product.

Accordingly, there is a need for perfusion bioreactor systems and methods that have lower perfusion rate yet have high recombinant protein productivity.

### SUMMARY

In one aspect, a perfusion bioreactor culture system is provided having a bioreactor and a cell retention device. The perfusion bioreactor culture system comprises a starting perfusion rate, a starting bioreactor volume, and a starting cell retention device volume. The system relates to decreasing the starting perfusion rate, resulting in increased residence time of the cells in the bioreactor and the cell retention device, and concomitantly increasing the starting bioreactor volume or decreasing the starting cell retention volume, or both. The system relates to varying the perfusion rate, bioreactor working volume or CRD working volume so as to achieve optimal residence time of cells in the conditions of the CRD.

In another aspect, a method of optimizing a perfusion bioreactor system is provided. The method comprises providing tissue culture fluid (also referred to herein as tissue culture media or medium) containing cells to a bioreactor system comprising a bioreactor and a cell retention device, wherein the system has a starting perfusion rate, a starting bioreactor volume, and a starting cell retention device volume, and decreasing the starting perfusion rate, resulting in increased residence time of the cells in the bioreactor and the cell retention device, and increasing the starting bioreactor volume or decreasing the starting cell retention volume, or both. The method relates to varying the perfusion rate, bioreactor working volume or CRD working volume so as to achieve optimal residence time of cells in the conditions of the CRD.

In another method aspect, a method of optimizing a perfusion bioreactor system is provided. The method comprises providing a first tissue culture fluid containing cells to a bioreactor system comprising a bioreactor and a cell retention device, the system having a starting perfusion rate, a starting bioreactor device volume, and a starting cell retention volume; decreasing the starting perfusion rate, resulting in increased residence time of the cells in the bioreactor and the cell retention device, and substituting the first tissue culture fluid for a second tissue culture fluid that has, compared to the first tissue culture fluid, adjustments of the of individual components of the cell culture by substitution or concentration changes.

In another method aspect, a method of optimizing a perfusion bioreactor system is provided. The method comprises providing a first tissue culture fluid containing cells that express a recombinant protein to a bioreactor system comprising a bioreactor and a cell retention device, wherein the system has a starting perfusion rate, a starting bioreactor volume, and a starting cell retention device volume, decreasing the starting perfusion rate, resulting in increased residence time of the cells in the bioreactor and the cell retention device, and adding a stabilizer of the recombinant protein to reduce degradation.

These and other features of the present teachings are set forth herein.

### DRAWINGS

The skilled artisan will understand that the drawings, described below, are for illustration purposes only. The drawings are not intended to limit the scope of the present teachings in any way.

FIG. 1 shows a schematic embodiment of a perfusion bioreactor system.

FIG. 2 shows a graph of viable cell density (diamond) and relative CSPR (square) in the Y-axis along the 1 L perfusion culture (X-axis, in days), for stepwise reduction in CSPR. CSPR is given in relative units.

FIG. 3 shows a graph of viable cell density (VCD, diamond) and potency (square), shown as normalized potency, of samples from the 1 L perfusion cell culture with stepwise reduction of CSPR.

FIGS. 4A-B show a bar (A) and graph (B) of observed mean potency difference (in %) relative to calculated potency at different CSPRs. Calculated potency is set at 100%.

FIG. 5 shows a graph of metabolism data for glucose and lactate, during the 1 L perfusion cell culture with stepwise reduction in CSPR Time frames (in days) with relative changes in CSPR are indicated.

FIG. 6 shows a graph of decrease in FVIII activity in the supernatant (spent media/harvested culture fluid): Experiment, Incubation at 37° C. for 9 hours. Residual FVIII activities are shown in percent of control.

FIG. 7 shows a graph of comparison of calculated FVIII activity using data from FVIII stability tests and experimentally determined activity from the CSPR reduction experiment. Calculated titer at the different CSPR levels are given in % with 100% being the initial potency of nascent FVIII.

FIGS. 8A-B show graphs of viable cell density and targeted CSPR rates (A) and FVIII potency in bioreactor samples (B) using different ratios of bioreactor and cell retention device.

FIGS. 9A-B show graphs of Glutamine and Glutamate. Concentrations in samples (A) and specific growth rate of FVIII producing cells (B).

FIGS. 10A-B show graphs of productivity of bioreactor system at different CSPRs and bioreactor working volumes (A) and calculated productivity per 1 L culture at different culture CSPRs (B).

FIGS. 11A-B show that added stabilizer can (dose-dependently) reduce potency loss (˜13-15%) due to residence time increase in bioreactor but does not compensate for total loss (˜23%).

FIG. 12 shows a flowchart illustrating a method of optimizing perfusion bioreactor system according to the embodiments.

FIG. 13 shows another flowchart illustrating another method of optimizing perfusion bioreactor system according to the embodiments.

FIG. 14 shows yet another flowchart illustrating another method of optimizing perfusion bioreactor system according to the embodiments.

### DESCRIPTION OF VARIOUS EMBODIMENTS

Embodiments of the invention provide methods and systems for increasing production capacity of perfusion cell culture system.

Reducing perfusion rate increases the cell (and recombinant protein/FVIII product) residence time in the CRD as well as in the bioreactor, resulting in decreased production of active recombinant protein product, such as FVIII. In certain embodiments, the reduction in perfusion rate is compensated by changing the relative volumes of the bioreactor to CRD. In some embodiments, the change in volume is in about the same proportion as the reduction in perfusion rate. For example, a reduction in perfusion rate in half is accomplished by concomitantly doubling of the volume-ratio of the bioreactor to CRD. The systems and methods according to embodiments of the invention may result in robust production of recombinant protein products. Decrease in perfusion rate can also be compensated by adjustments in components of the tissue culture media, or by adding a stabilizer (such as calcium for recombinant FVIII, i.e., rFVIII) to reduce degradation of the protein product(s).

The perfusion cell culture system includes two key unit operations: the bioreactor, where conditions are generally optimal for recombinant protein production (such as rFVIII) and the CRD (e.g., a settler), where conditions are not optimal to recombinant protein product/rFVIII production due to lack of oxygen control and a generally low operating temperature compared to the physiological temperature in the bioreactor. Thus, the cell culture continuously circulates through tubing between environments that are conducive to, and less conducive to, cellular productivity and recombinant protein product/rFVIII production. Moreover, the longer the residence times of the cells within the CRD relative to the bioreactor, the larger the expected loss in productivity due to transition of cells from a lower to higher cell metabolic state.

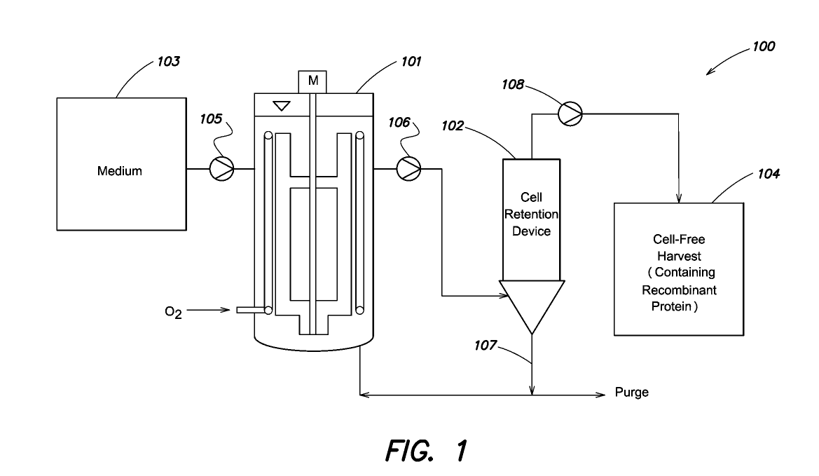

FIG. 1 illustrates a block diagram of an embodiment of a perfusion bioreactor culture system 100. The perfusion bioreactor culture system 100 comprises a bioreactor 101 having a bioreactor inlet 105 and a bioreactor outlet 106. The bioreactor 101 comprises a culture chamber configured to hold a tissue culture fluid (TCF) and cells to be cultured. The perfusion bioreactor culture system 100 comprises a cell retention device (CRD) 102, which could comprise a cell aggregate trap or other suitable cell separator. The cell retention device 102 has an outlet 107 for recirculating the tissue culture fluid and the cells to the bioreactor 101. The cell retention device 102 also has another outlet 108, which sends a harvest output of tissue culture fluid with only a small amount of cells to cell-free harvest 104 for the isolation and purification of the recombinant protein product. The perfusion bioreactor culture system 100 also comprises a medium vessel 103, which sends in fresh tissue culture fluid to the bioreactor via inlet 105. The perfusion bioreactor system 100 can be used for the production of biologics such as coagulant factors. For example, the perfusion bioreactor culture system 100 and methods described herein can be used to manufacture any protein product, including recombinant protein product and including coagulant factors such as Factor VII, VIII, or Factor IX, or other suitable factors or substances.

In a system embodiment, a perfusion bioreactor culture system 100 is provided. This system comprises: a bioreactor 101 configured to contain a tissue culture fluid and cells to be cultured; a CRD 102 configured to receive tissue culture fluid containing cells from the bioreactor 101, separate some cells from the tissue culture fluid and provide harvest output of tissue culture fluid and cells, and provide a recirculation output of tissue culture fluid and cells to the bioreactor 101. The system 100 has a starting perfusion rate (a first perfusion rate), a starting bioreactor volume (a first bioreactor volume), a starting cell retention device volume (a first starting cell retention device volume), and a starting volume ratio of the starting bioreactor volume and a starting cell retention device volume (a first volume ratio). In one or more embodiments, the starting perfusion rate is decreased (to a second perfusion rate), resulting in increased residence time of the cells in the bioreactor 101 and the cell retention device 102. Additionally or alternatively, the starting bioreactor volume is increased (to a second bioreactor volume) or the starting cell retention device volume is decreased (to a second cell retention device volume), or both, resulting in an increase in the starting volume ratio (to a second volume ratio).

In one or more embodiments, the increase in the starting volume ratio is in about the same proportion as the reduction in the starting perfusion rate. In certain embodiments, the starting perfusion rate is decreased in a range of from about a third to about two thirds. In other embodiments, the starting perfusion rate is decreased by up to about a third. In other embodiments, the starting perfusion rate is decreased by up to about a half, and in yet other embodiments, the starting perfusion rate is decreased by up to about two thirds. In some embodiments, the starting bioreactor volume is increased by about a third to about two thirds; in other embodiments, the starting bioreactor volume is increased by up to about a third. In other embodiments, the starting bioreactor volume is increased by up to about a half, and yet in other embodiments, the starting bioreactor volume is increased by up to about two thirds.

In one or more embodiments, the starting cell retention device volume is decreased by about a third to about two thirds. In some embodiments, the starting cell retention device volume is decreased by up to about a third. In some embodiments, the starting cell retention device volume is decreased by up to about a half, and yet in other embodiments, the starting cell retention device volume is decreased by up to about two thirds.

In one or more embodiments, the starting volume ratio is increased by about a third to about two thirds. In some embodiments, the starting volume ratio is increased by up to about a third. In some embodiments, the starting volume ratio is increased by up to about a half, and yet in other embodiments, the starting volume ratio is increased by up to about two thirds. In certain embodiments, the starting perfusion rate is about 1 to 15 volumes per day.

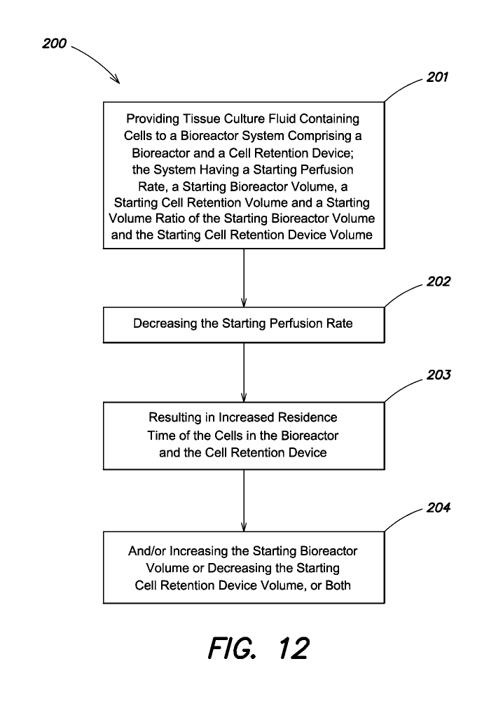

Methods of optimizing a perfusion bioreactor culture system 100 will now be described with reference to FIG. 12. One method 200 of optimizing a perfusion bioreactor culture system 100, comprises, in 201, providing tissue culture fluid containing cells to a bioreactor system comprising a bioreactor and a cell retention device, the system having a starting perfusion rate (a first perfusion rate), a starting bioreactor volume (a first bioreactor volume), a starting cell retention device volume (a first cell retention device volume), and a starting volume ratio of the starting bioreactor volume and the starting cell retention device volume (a first volume ratio). The method further comprises, in 202, decreasing the starting perfusion rate (to a second perfusion rate), resulting, in 203, in increased residence time of the cells in the bioreactor and the cell retention device, and/or, in 204, either increasing the starting bioreactor volume (to a second bioreactor volume) or decreasing the starting cell retention volume (to a second cell retention volume), or both, resulting in an increase in the starting volume ratio (to a second volume ratio).

In some embodiments, the increase in the starting volume ratio is in about the same proportion as the reduction in the starting perfusion rate. In some embodiments, the starting perfusion rate is decreased in a range of from about a third to about two thirds. In other embodiments, the starting perfusion rate is decreased by up to about a third. In other embodiments, the starting perfusion rate is decreased by up to about a half, and in yet other embodiments, the starting perfusion rate is decreased by up to about two thirds.

In certain embodiments, the starting bioreactor volume is increased by about a third to about two thirds. In some embodiments, the starting bioreactor volume is increased by up to about a third. In other embodiments, the starting bioreactor volume is increased by up to about a half, and yet in other embodiments, the starting bioreactor volume is increased by up to about two thirds.

In other embodiments, the starting cell retention device volume is decreased by about a third to about two thirds. In some embodiments, the starting cell retention device volume is decreased by up to about a third. In other embodiments, the starting cell retention device volume is decreased by up to about a half, and yet in other embodiments, the starting cell retention device volume is decreased by up to about two thirds.

In some embodiments, the starting volume ratio is increased by about a third to about two thirds. In some embodiments, the starting volume ratio is increased by up to about a third. In other embodiments, the starting volume ratio is increased by up to about a half, and yet in other embodiments, the starting volume ratio is increased by up to about two thirds. In certain embodiments, the starting perfusion rate is about 1 to 15 volumes per day.

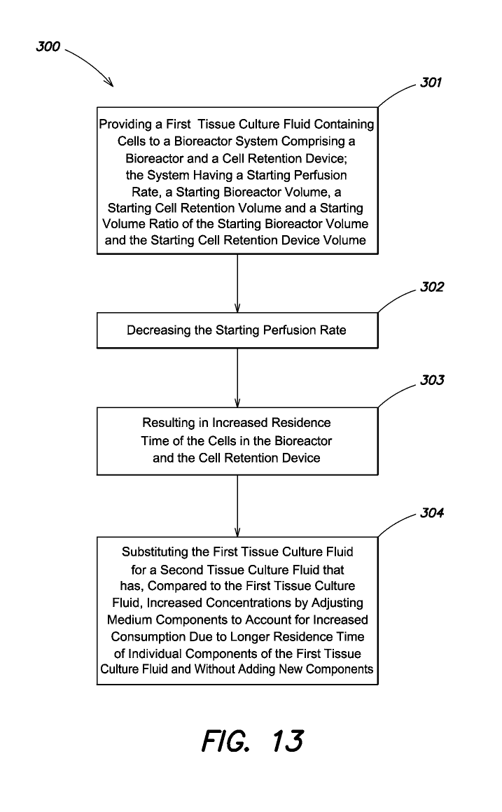

Another method of optimizing a perfusion bioreactor culture system 100 will now be described with reference to FIG. 13. One method 300 of optimizing a perfusion bioreactor culture system 100 comprises, in 301, providing a first tissue culture fluid containing cells to a bioreactor system comprising a bioreactor and a cell retention device, wherein the system has a starting perfusion rate (a first perfusion rate), a starting bioreactor volume, and a starting cell retention device volume. Furthermore, the method 300 comprises, in 302, decreasing the starting perfusion rate (to a second perfusion rate). This results, in 303, in increased residence time of the cells in the bioreactor and the cell retention device. The method 300 further comprises, in 304, substituting the first tissue culture fluid for a second tissue culture fluid that has, compared to the first tissue culture fluid, increased concentrations of individual components of the first tissue culture fluid and without adding new components. For example, the increased concentrations may include increasing the concentrations in a range from about 1 to 10 times of individual components of the first tissue culture fluid, or in a range from about 1.2 to about 5 times of individual components of the first tissue culture fluid, and cystine can be replaced with cysteine.

In some embodiments, the first tissue culture fluid can include amino acids, which can include, for example, any of the naturally occurring amino acids. In some embodiments, the second tissue culture fluid can have increased concentration of one or more of the amino acids, such as increases of in a range from about 1.1 to about 10 times the concentration present in the first tissue culture fluid. In some embodiments, the second tissue culture fluid can have increased concentration of one or more of the amino acids in a range from about 1.2 to about 5 times, or even about 1.2 to about 2 times the concentration present in the first tissue culture fluid. In some embodiments, the amino acids that are increased can be in a range from about 50% to about 75% of all of the amino acids present in the first tissue culture fluid. In some embodiments, the amino acid cystine can be replaced by additional cysteine, such that the second tissue culture fluid has about 1.1 to about 12 times more cysteine than the first tissue culture fluid. Other concentration ranges and/or percentages can be employed.

In some embodiments, the first tissue culture fluid can include salts, which can include potassium chloride, magnesium sulfate, sodium chloride, sodium phosphate, magnesium chloride, cupric sulfate, ferrous sulfate, zinc sulfate, ferric nitrate, selenium dioxide, calcium chloride and/or other salts that can be found in a tissue culture fluid. In some embodiments, the second tissue culture fluid can have increased concentration of one or more of the salts in a range from about 1.1 to about 10 times the concentration present in the first tissue culture fluid. In other embodiments, the second tissue culture fluid can have increased concentration of one or more of the salts in a range from about 1.2 to about 5 times or from about 1.2 to about 2 times the concentration present in the first tissue culture fluid. In some embodiments, the salts that are increased can be in a range from about 50% to about 75% of all of the salts present in the first tissue culture fluid. Other concentration ranges and/or percentages can be employed.

In some embodiments, the first tissue culture fluid can include vitamins, which can include biotin, choline chloride, calcium pantothenate, folic acid, hypoxanthine, inositol, niacinamide, vitamin C, pyridoxine, riboflavin, thiamine, thymidine, vitamin B-12, pyridoxal, putrescine, and/or other vitamins that can be found in a tissue culture fluid. In some embodiments, the second tissue culture fluid can have increased concentration of one or more of the vitamins in a range from about 1.1 to about 5 times the concentration present in the first tissue culture fluid. In other embodiments, the second tissue culture fluid can have increased concentration of one or more of the vitamins in a range from about 1.2 to about 3 times the concentration present in the first tissue culture fluid. In some embodiments, the vitamins that are increased can be in a range from about 50% to about 75% of all of the vitamins present in the first tissue culture fluid. Other concentration ranges and/or percentages can be employed.

In some embodiments, the first tissue culture fluid can include one or more components other than those listed above (“other components”), which can include dextrose, mannose, sodium pyruvate, phenol red, glutathione, linoleic acid, lipoic acid, ethanolamine, mercaptoethanol, ortho phophorylethanolamine and/or other components that can be found in a tissue culture fluid. In some embodiments, the second tissue culture fluid has increased concentration of one or more of the “other components” in a range from about 1.1 to about 10 times the concentration present in the first tissue culture fluid. In some embodiments, the second tissue culture fluid has increased concentration of one or more of the “other components” in a range from about 1.2 to about 5 times or about 1.2 to about 2 times the concentration present in the first tissue culture fluid. In some embodiments, the one or more “other components” that are increased can be in a range from about 50% to about 75% of all of the “other components” present in the first tissue culture fluid. Other concentration ranges and/or percentages can be employed.

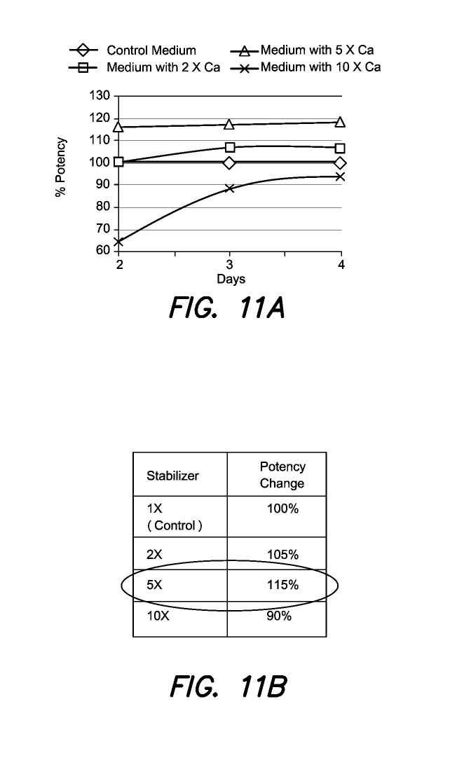

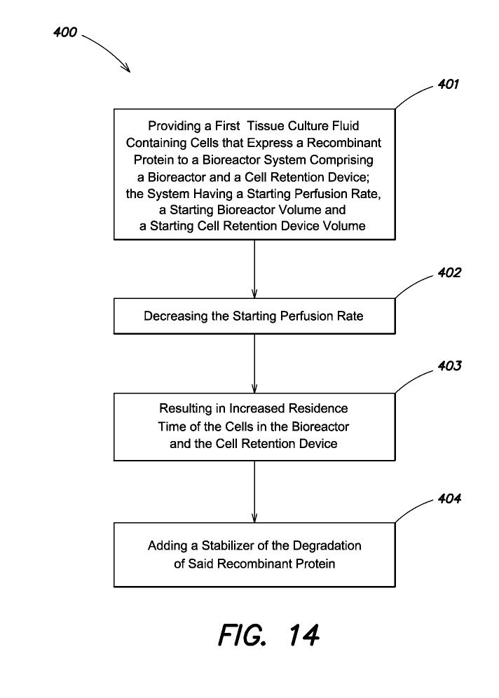

Another method of optimizing a perfusion bioreactor culture system 400 will now be described with reference to FIG. 14. The method 400 of optimizing a perfusion bioreactor system 100 comprises, in 401, providing a first tissue culture fluid containing cells that express a recombinant protein to a bioreactor system comprising a bioreactor and a cell retention device, the system having a starting perfusion rate (a first perfusion rate), a starting bioreactor volume, and a starting cell retention device volume. The method 400 further comprises, in 402, decreasing the starting perfusion rate (to a second perfusion rate), resulting, in 403, in increased residence time of the cells in the bioreactor and the cell retention device. The method 400 also comprises, in 404, adding a stabilizer to mitigate the degradation of the recombinant protein. In certain embodiments, the stabilizer is calcium. As shown in FIGS. 11A-11B, adding stabilizer reduces potency loss (˜13-15%) due to residence time increase in bioreactor.

Example perfusion culture systems for the production of Factor VIII are described, for example, in U.S. Pat. No. 6,338,964 entitled “Process and Medium For Mammalian Cell Culture Under Low Dissolved Carbon Dioxide Concentration,” and in Boedeker, B.G.D., Seminars in Thrombosis and Hemostasis, 27(4), pages 385-394, and in U.S. Application No. 61/587,940, filed Jan. 18, 2012, the disclosures of all of which are hereby incorporated by reference in their entirety herein. The bioreactor 101 and the cell retention device 102 are known in the art. In certain embodiments, the cell retention device 102 can further comprise a cell aggregate trap configured to receive the recirculation output of tissue culture fluid and cells, separate cell aggregates from the recirculation output of tissue culture fluid and cells, and return the remaining tissue culture fluid and cells to the bioreactor 101.

Cell cultivation can be started by inoculating with cells from previously-grown culture. Typical bioreactor parameters can be maintained (e.g., automatically) under stable conditions, such as at a temperature at about 37° C., pH of about 6.8, dissolved oxygen (DO) of about 50% of air saturation, and approximately constant liquid volume. Other bioreactor parameters can be used. DO and pH can be measured on-line using commercially-available probes. The bioreactor process can be started in batch or fed batch mode for allowing the initial cell concentration to increase. This can be followed by a perfusion stage wherein the cell culture medium is pumped continuously into the bioreactor 101 through inlet 105 and the tissue culture fluid containing cells are pumped out through outlet 106. A flow rate of tissue culture fluid can be controlled and increased proportionally with the cell concentration. A steady state or stable perfusion process can be established when the cell concentration reaches a target level (e.g., greater than 1×10⁶ cells/mL) in the bioreactor 101 and can be controlled at this concentration. At this point, the flow rate can be held constant. The cell density can be held for example, between about 4 million to about 40 million cells per milliliter, in the perfusion bioreactor system 100.

Known downstream practices can be employed to purify the recombinant protein produced using systems and methods described herein. Typical purification processes can include cell separation, concentration, precipitation, chromatography, and filtration, or the like. Other purification processes are also possible.

The cells can be any eukaryotic or prokaryotic cells, including mammalian cells, plant cells, insect cells, yeast cells, and bacterial cells. The cells can be any cells making any biologic protein products. The cells could be recombinant cells that are engineered to express one or more recombinant protein products. The cells could be expressing antibody molecules. The product can be any protein product, including recombinant protein products such as coagulation factors, including for example factor VII, factor VIII, factor IX and factor X. In some embodiments, the cells are mammalian cells, such as, for example, BHK (baby Hamster kidney) cells, CHO (Chinese Hamster ovary) cells, HKB (hybrid of kidney and B cells) cells, HEK (human embryonic kidney) cells, and NS0 cells. The mammalian cells can be recombinant cells expressing factor VIII.

The tissue culture fluid, also known as tissue culture media, can be any suitable type of tissue culture media. For example, the tissue culture fluid can be a media composition based on a commercially available DMEM/F12 formulation manufactured by JRH (Lenexa, Kans.) or Life Technologies (Grand Island, N.Y.) supplied with other supplements such as iron, Pluronic F-68, or insulin, and can be essentially free of other proteins. Complexing agents such as histidine (his) and/or iminodiacetic acid (IDA) can be used, and/or organic buffers such as MOPS (3-[N-Morpholino]propanesulfonic acid), IFS (N-tris[Hydroxymethyl]methyl-2-aminoethanesulfonic acid), BES (N,N-bis[2-Hydroxyethyl]-2-aminoethanesulfonic acid) and/or TRIZMA (tris[Hydroxymethyl]aminoethane) can be used; all of which can be obtained from Sigma (Sigma, St. Louis, Mo.), for example. In some embodiments, the tissue culture fluid can be supplemented with known concentrations of these complexing agents and/or organic buffers individually or in combination. In some embodiments, a tissue culture fluid can contain EDTA, e.g., 50 μM, or another suitable metal (e.g., iron) chelating agent. Other compositions, formulations, supplements, complexing agents and/or buffers can be used.

The starting perfusion rate can be, for example, a perfusion rate set by the biological license of a biologic product approved by the FDA. The starting perfusion rate can be, for example, one that is thought to be optimized. The starting bioreactor volume and starting cell retention device volume can also be, for example, those set in the biological license of a biologic product or is otherwise considered optimized for a particular system. The starting perfusion rate, the starting bioreactor volume, or cell retention device volume can also be, for example, those recommended by the manufacturer of the systems. Note that a starting perfusion rate, starting bioreactor volume and/or cell retention device volume need not be the actual values employed during operation. Rather, such starting values may simply be employed for selection of the perfusion rate, bioreactor volume and/or cell retention device volume employed during operation. The bioreactor volume and/or cell retention device volume can be operating, or working, volumes.

The residence time is the average time that the cells and the product are exposed to the conditions of the unit operations of the system 100. Two key unit operations are the bioreactor 101 and the cell retention device 102.

Aspects of the present teachings may be further understood in light of the following examples, which should not be construed as limiting the scope of the present teachings in any way.

### EXAMPLES

### Example 1

### Effects of Decreasing the Starting Perfusion Rate and Increasing Components of the Media

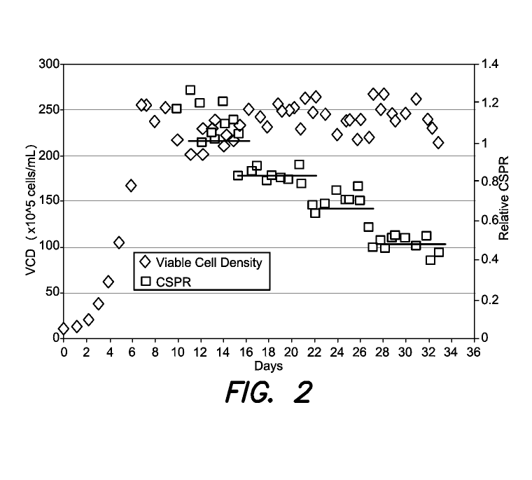

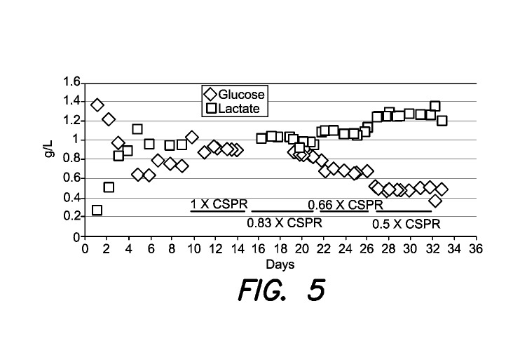

In this example, enriched media and a bioreactor vessel 101 operated at a 1 L working volume and equipped with a 375 mL settler-type cell retention device 102, for cell retention were used. BHK cells producing rhFVIII, an active ingredient of KG-FS, were grown until reaching steady state at a cell density of about 25×10⁶ cells/mL. In this embodiment, the starting perfusion rate (the control rate) was maintained at a high rate of 11 volumes/day for 5 days. Two systems were set up. In the experimental system, using the novel VM2 media, perfusion rate was stepwise reduced to 0.83, 0.67 and 0.5 fraction of the initial perfusion rate, by adjusting the harvest pump speed based on the measured cell density. The culture was kept at each perfusion rate level for 5 days and samples were collected for potency testing (Table 1). Cell viability (FIG. 2) and metabolism (FIG. 5) were not significantly affected by the change in perfusion rate. Lactate increased at the lower perfusion rates, but it also increased in the control bioreactor run at a perfusion rate of 11 volumes/D towards the latter part of the run (FIG. 5). Growth rate was apparently not impacted by the changes made to the perfusion rate either because purge rates did not change and because the viable cell density (VCD) remained constantly high along the perfusion rate-reduction experiment (FIG. 2). In another control system, a perfusion rate of 11 vol/day was maintained throughout the whole run (not shown). The collected samples were analyzed for FVIII activity.

TABLE 1
Target perfusion rates of test and control system

| | System 1 | System 2 |
|---|---|---|
| Time period | VM2 media | R3 production media |
| day 1 to day 10 | Growth until steady | Growth until steady |
| | state | state |
| day 10 to day 15 | Perfusion rate 11 vol/d | Perfusion rate 11 vol/d |
| day 15 to day 21 | Perfusion rate 9 | Perfusion rate 11 vol/d |
| | vol/day | |
| day 21 to day 26 | Perfusion rate 7.3 vol/d | Perfusion rate 11 vol/d |
| day 26 to day 31 | Perfusion rate 5.5 | Perfusion rate 5.5 |
| | vol/day | vol/day |

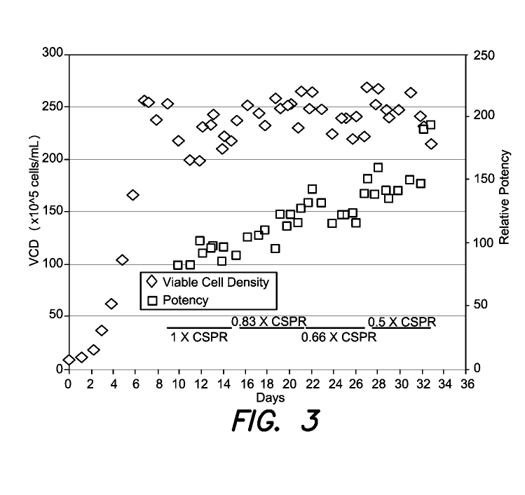

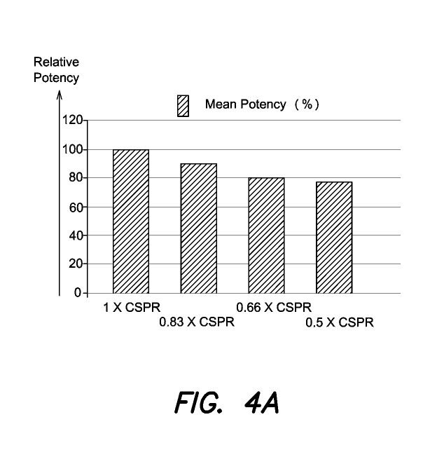

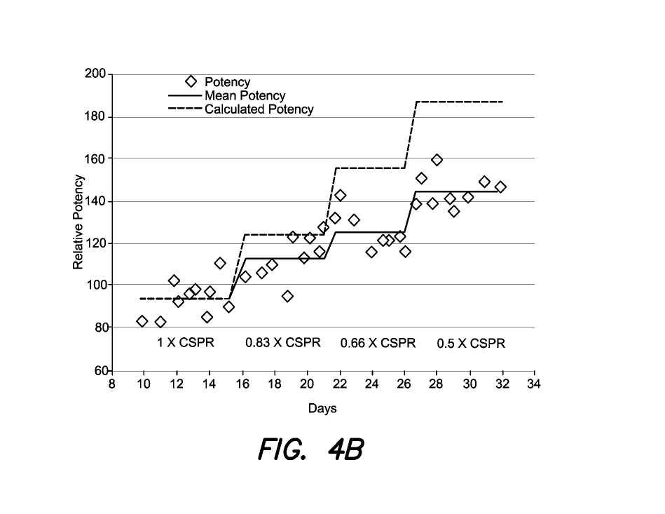

R3 is a modified DMEM-F12 (1:1) based medium and VM2 is an enriched DMEM-F12 based medium (include specific enhancements). As shown, with every step of perfusion rate reduction, FVIII titer increased (FIGS. 4A-4B). At a perfusion rate level of 5.5 vol/day, the mean potency was about 50% higher compared to that at initial perfusion rate of 11 vol/Day (FIG. 3). In the control fermenter, FVIII activity remained at a constant level (not shown). However, while potency increased by ˜50% when perfusion rate was reduced in half, it did not match the calculated potency, which should have been a 100% increase (i.e., double the potency, when reducing the perfusion rate in half)—in order to obtain the same output per unit operation.

The difference between measured and calculated values increased with every reduction step to about 23% less than expected at 5.5 vol/day (half of the normal perfusion rate, half of media volume as at normal perfusion rate) (FIGS. 4A-4B).

By reducing the perfusion rate by using half of the media volume (about half of media costs) with the novel VM2 media, compared to normal perfusion fermentation, there was about 50% more activity of FVIII in the harvest (instead of 100% more to give the same output).

A comparison between the observed titer and the calculated titer shows that the measured FVIII activity was lower compared to the calculated values. Productivity of the cell culture system was therefore found to be lower at lower perfusion rate rates.

### Example 2

### FVIII Stability

For the examination of the impact of residence time on destabilization of FVIII activity, fresh bioreactor samples from steady state perfusion cultures were used.

Cells were removed by centrifugation to avoid further production of FVIII and the supernatant was incubated under cell culture simulated conditions in roller tubes at 37° C. in an incubator.

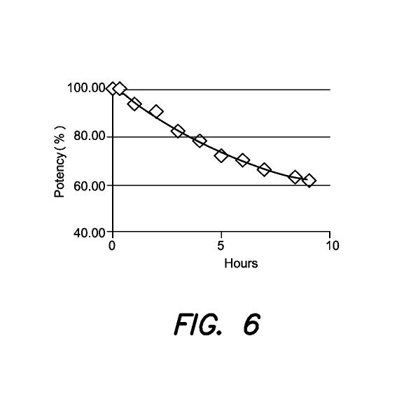

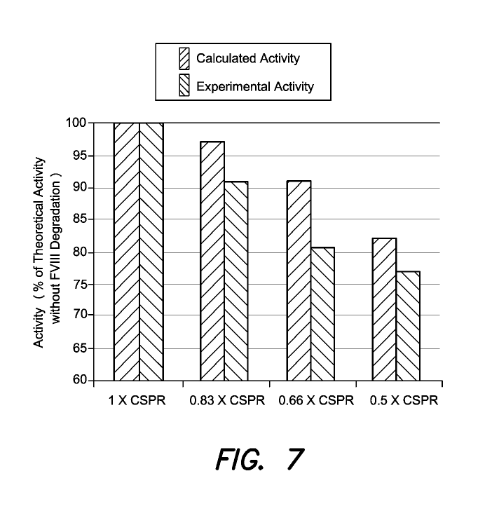

At defined time points, samples were taken for FVIII determination. The results showed a large decrease in FVIII activity from 100% to about 60% within the first day of incubation, and a slower decrease during further incubation (FIG. 6).

Evidently, increases in residence time unfavorably impacts FVIII activity.

Using the data from the time-dependent decrease in FVIII activity, the theoretical decrease of FVIII activity resulting from residence time increase during the perfusion rate reduction experiment (Example 1) were calculated and compared it to the experimental activity shown in FIG. 4A-4B. The comparison shows that the difference between the observed titer and the calculated titer could partly be the result of FVIII instability during the prolonged residence time at reduced perfusion rates (FIG. 6). However, FVIII stability loss does not account for the overall reduction in potency at reduced perfusion rates.

### Example 3

### Perfusion Rate Reduction Coupled to Increasing the Bioreactor Working Volume

Example 2 shows that perfusion rate reduction was limited by FVIII potency loss due to the longer residence time.

To overcome the negative effect of prolonged residence time, an increase of the ratio of the bioreactor working volume to the cell retention device volume (e.g., settler volume) was tested.

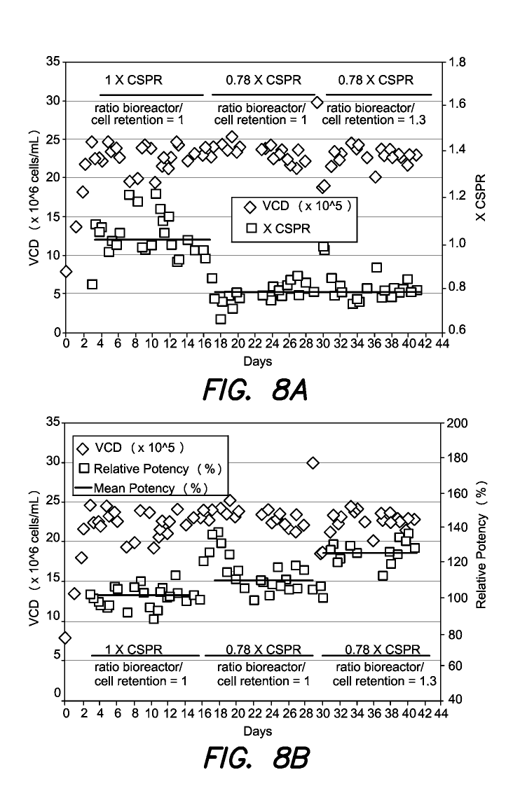

A perfusion culture was carried out with perfusion rate reduction coupled to working volume increase as summarized in Table 2. Cells were grown to steady state cell density of about 24×10⁶ cells/ml within about 3 days after inoculation with 9×10⁶ cells/mL. After collecting a data set at normal perfusion rate of 11 vol/day (1×) for about 14 days (time period 1), perfusion rate was targeted at 8.5 vol/d (0.78×) for 12 days by reducing the harvest flow rate and keeping a constant cell density of about 24×10⁶ cells/mL (time period 2). For the following 12 days of cell culture, the working volume of the bioreactor 101 was increased from 1 L to 1.3 L by adjustment to the level sensor (time period 3). Cell density was kept at 24×10⁶ cells/mL and perfusion rate targeted at 8.5 vol/d (Table 2, FIG. 8A).

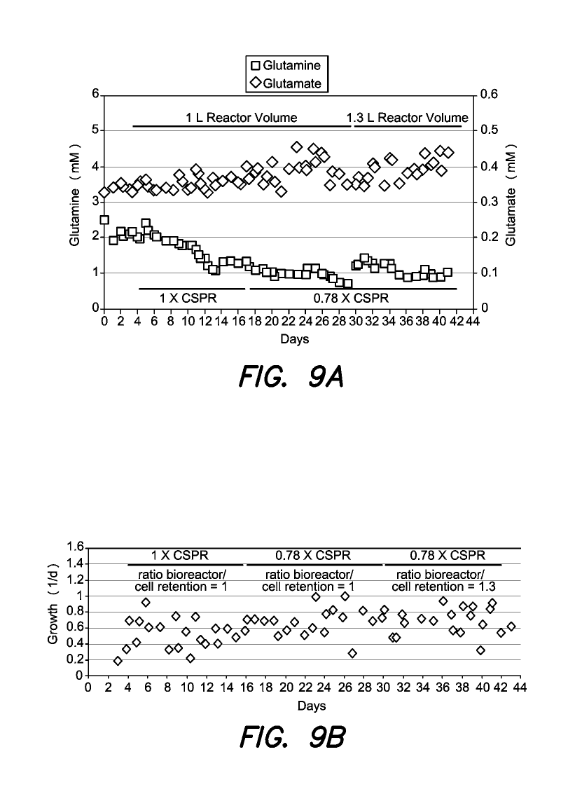

Standard DMEM-F12 based production media was used in this example, which apparently contains sufficient nutrients for normal cell culture performance at the perfusion rates tested. Glucose concentrations remained above 0.8 g/L during reduced perfusion rate and glutamine concentrations were about 1 mM during period where the Perfusion rate was 8.5 vol/day (0.78×). No impact to cell growth rate was apparent upon lowering the perfusion rate or increasing the working volume of the bioreactor (FIG. 9).

TABLE 2
Target perfusion rate and working volume of bioreactor

| | | | working vol. |
|---|---|---|---|
| | | | Ratio |
| | | perfusion | bioreactor/cell |
| | | rate | retention |
| Time period | Time period | (vol/day) | device |
| | day 1 to | Growth until | |
| | day 3 | steady state | |
| time period 1 | day 3 to | 11 | 1 Liter |
| | day 17 | | |
| time period 2 | day 17 to | 8.5 | 1 Liter |
| | day 29 | | |
| time period 3 | day 29 to | 8.5 | 1.3 Liter |
| | day 41 | | |

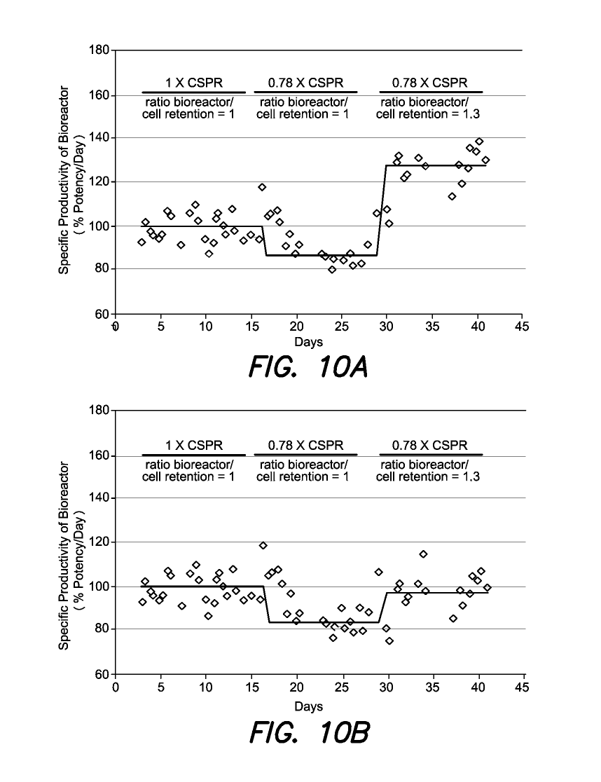

FVIII activities of samples were about 10% higher after reducing the perfusion rate from 11 vol/day (1×) to 8.5 vol./day (0.78×, FIG. 8B). The calculated productivity of the system was decreased to about 86% of the productivity during time period 1, (FIGS. 10A-10B, Table 1). This was in accordance with Example 2 (see FIGS. 4A-4B).

In time period 3, the working volume ratio of the working volume of the bioreactor 101/the working volume of the CRD 102 was increased from 1× to 1.3×, while maintaining the reduced perfusion rate of 0.78× and thus increasing the ratio of culture volume to CRD volume, resulting in reduction of culture residence time in the CRD 102 and loss of cellular productivity.

Indeed, FVIII activity increased during this time period (see FIGS. 10A-10B).

The calculated system's productivity showed an increase of 127% compared to the productivity of the system with 1× working volume and perfusion rate of 11 vol/day (1×). This is close to the calculated productivity of 130% for the 1.3× working volume (FIGS. 10A-10B, Table 3).

Normalized to 1× culture volume, the calculated productivity of time period 3 was about the same as the productivity of the culture under standard conditions (98% vs. 100%, Table 3).

This demonstrates that it is feasible to reduce the Cell-specific Perfusion Rate CSPR by at least 30% while maintaining cell-specific and overall system productivity because the concentration of FVIII in the harvest increased proportionally.

TABLE 3
Productivities at different cell culture
CSPRs and bioreactor/Cell Retention Device working volumes

| | | | Mean | Mean |
|---|---|---|---|---|
| Working | perfusion | | Productivity | productivity |
| volume | rate | Residence | per reactor | per 1 L |
| (L) | (vol/d) | time (h) | (%) | culture (%) |
| 1 | 11 | 3.06 | 100 | 100 |
| 1 | 8.5 | 3.93 | 85.9 | 85.9 |
| 1.3 | 8.5 | 3.68 | 127.4 | 98 |

The 11 vol/day and 8.5 vol/day correspond to 1× and 0.78×, respectively; Cell density was approximately: 24×10⁶ cells/mL. The total residence time of FVIII is composed of the residence times in the productive bioreactor (Tₚᵣ in bioreactor volume Vₚᵣ) and in the non-productive settler (Tₙₚᵣ in settler volume Vₙₚᵣ). Thus, the mean residence time (T_{R}) for FVIII is as follows (V_{media}: total volume of media per 24 hours):

T_{R}=Tₚᵣ+Tₙₚᵣ=Vₚᵣ/V_{media}×24 hours+Vₙₚᵣ/V_{media}×24 hours

In Table 4, the residence times of the different fermentation conditions are shown. The productivity correlates inversely proportional with Tₙₚᵣ. The effect of Tₚᵣ increase seems to have less influence on productivity.

Tₙₚᵣ of the current FVIII production system is due to the smaller settler/bioreactor volume; only about half of Tₙₚᵣ of the 1 L working volume system using the same perfusion rate of 11 vol/day and cell density.

TABLE 4
Comparison of FVIII residence times at
different FVIII fermentation conditions

| | | | | | Mean |
|---|---|---|---|---|---|
| Working volume | | | | | productivity |
| ratio | | | | | normalized |
| bioreactor/cell | | | | T_{R} (Total | to 1 L |
| retention | perfusion | | Tₙₚᵣ | residence | culture |
| device (X) | rate (X) | Tₚᵣ (h) | (h) | time) (h) | system (%) |
| 1 | 1 | 2.22 | 0.83 | 3.06 | 100 |
| 1 | 0.78 | 2.86 | 1.07 | 3.93 | 85.9 |
| 1.3 | 0.78 | 2.86 | 0.82 | 3.68 | 98 |

Assuming a cell density of 24 × 10⁶ cells/mL.

### Example 4

### Material and Methods for Examples 1-3

### Perfusion Cell Cultures

For scale up, recombinant BHK cells expressing recombinant human FVIII, an active ingredient of KG-FS, were inoculated in shake flasks using R3 production media. Flasks were incubated at 35.5° C. and 30 rpm and successively split until the desired amount of cells was present.

Cells from scale up were inoculated at 9×10⁶ vc/mL into a 1.5 L DASGIP vessel at a working volume of 1 L on a DASGIP control station. The working volume was kept constant by a level sensor, which controlled the media pump.

Perfusion was established using a CRD (e.g., cell settler of 0.375 mL volume) at a target CSPR of 7.3 vol/day during cell accumulation and 11 vol/day at steady state by adjustment of the harvest pump dependent on the measured cell density. Perfusion rates were calculated from the pre-calibrated harvest pump but were also checked by measuring harvest volume. Actual perfusion rate was consistently equal to the volume predicted by the calibration. Temperature was controlled at 35.5° C. using the station thermostat and the CRD temperature was controlled at 20-23° C. by cooling of the tubing leading to the CRD in a refrigerated water bath set at 16-18° C. Aeration was provided by a silicone tube aerator with oxygen percentage in the gas controlled by the dissolved oxygen controller. Typical oxygen percentage during steady state was 70% to 80%. Back pressure was kept at 0.5 to 0.6 bar. Cell density at steady state was targeted at 25×10⁶ vc/mL and controlled to maintain dissolved oxygen sufficiency. Supplementary aeration was provided by head space aeration of 5 L/hour. Culture pH was controlled at a target of 6.85 by addition of 4% sodium carbonate solution.

For the reduction of perfusion rate the harvest pump was set to the appropriate pump rate, while cell density was kept constant. Oxygen supply was adjusted to meet control set points.

If necessary, the increase of the working volume ratio from 1× to 1.3× was accomplished by pulling the level sensor to the appropriate position. Oxygen supply was adjusted by increasing the oxygen percentage in the gas mix to maintain the cell density at the required level.

Samples of the cell culture were withdrawn from the reactor vessel using an external sample pump (Watson Marlow 101U/R, Watson Marlow, Inc., Wilmington, Mass.) and were analyzed using a cell counting system (Cedex XS analyzer, Innovatis, UK) on cell density and viability, and two YSI 2700s (one measuring glucose and lactate, and another glutamine and glutamate). Factor VIII in the samples was stabilized by addition of Calcium (to 20 mM), frozen at −70 degrees C. and later analyzed for rFVIII (recombinant FVIII) potency by a chromogenic assay.

The chromogenic potency assay method includes two consecutive steps where the intensity of color is proportional to the Factor VIII activity in the sample. In the first step, Factor X is activated to Factor Xa by Factor IXa with its cofactor, Factor VIIIa, in the presence of optimal amounts of calcium ions and phospholipids. Excess amounts of Factor X are present such that the rate of activation of Factor X is solely dependent on the amount of Factor VIII. In the second step, Factor Xa hydrolyzes the chromogenic substrate to yield a chromophore and the color intensity is read photometrically at 405 nm. Potency of an unknown is calculated and the validity of the assay is checked using the linear regression statistical method. Activity is reported in International Units per mL (IU/mL).

### FVIII Stability Tests

Fourteen mL of cell-free (centrifuged) culture supernatant was collected from 1 L working volume of perfusion cultures grown in normal R3 media at a cell specific perfusion rate of 11 vol/d and transferred to 50 mL rolling tubes with vented caps. A sample of the supernatant was frozen with 20 mM calcium serving as a control. The tubes were incubated at 37° C. at 5% CO2 and 80% humidity at 30 rpm. At defined time points samples were taken, calcium was added as needed to bring all samples to a final concentration of 20 mM, and were stored at −80° C. until tested for FVIII activity. All experiments were carried out in duplicates.

### Media Formulations

### Design of Enriched Media VM2

For VM2 media, most of the components were used at 2× concentrations. Changes, relative to standard R3 media which is based on DMEM/F12 at a 1:1 ratio, were as follows. The concentrations of amino acids were determined based on their consumption rate, calculated in spent media analysis experiments. The low soluble cystine was replaced with a higher concentration of (the more soluble) cysteine. Glutamine was included at 10 mM (2× of the R3 media concentration). Magnesium was used at the same concentration as in standard R3 media, and trace elements were used at 2× concentrations, with the exception of selenium dioxide, which was used at 1×. Calcium was included at 2× concentration. Glucose and mannose were kept at 1 g/L, and 3 g/L, respectively, i.e., the same as in the standard R3 medium; glutamine concentration was set to 10 mM. Oleic acid, cholesterol, insulin and any other additives were also used at the same concentrations as in normal R3 (DMEM/F12 1:1) medium. Importantly, no new media components (not present in the R3 modified DMEM/F12 medium) were introduced in VM2—only the concentrations of specific components, have been altered.

### Concluding Remarks Regarding Examples 1-4

Enriched media formulation was designed in order to maintain sufficient nutrition levels at CSPR levels of about half of the CSPR rate of 11 vol/d used in FVIII production. It was shown that CSPR levels can be reduced from 11 to 8.5 vol/day, using normal R3 (DMEM/F12 based) production media nutrition. This shows that nutrient limitation and/or by-product toxic waste accumulation are not limiting at the reduced CSPR tested.

At reduced perfusion rates, while FVIII potency increased, the increase was lower than calculated, assuming the same cell specific productivity.

FVIII stability experiments show that longer residence time in the cell culture system leads to FVIII potency loss, presumably due to degradation. The decrease of FVIII activity in (cell-free) stability experiments only partially explains the gap with the theoretical FVIII potency during CSPR reduction.

The volume ratio bioreactor/CRD of the current 1L working volume perfusion system is 2.67. With the increase of the bioreactor/CRD working volume to 1.3, the volume ratio increased to 3.47.

By changing the ratio of bioreactor to CRD volume, the productivity of cells in perfusion culture was increased at a CSPR of 8.5 vol/day close to the same level as the productivity of the system at a CSPR of 11 vol/d.

From the economic point of view, this would mean cost savings in the upstream process with reduced fresh media volume as well as in the downstream process with lower harvest volume by at least a factor of 1.3.

The residence time TR of FVIII containing media is distributed in Tpr and Tnpr. The examples above demonstrate that mainly Tnpr influences the productivity of the system.

Thus another strategy for optimization of productivity could be the minimization of Tnpr by minimizing the volumes of the CRD (e.g., settler) and tubings coupled thereto.

Glutamine concentrations (using R3 media at CSPR 8.5.vol/d) were above 0.6 mM, which in prior studies was the concentration below which growth rate becomes limited. No growth limitations were observed under the described conditions with a cell density of about 24×10⁶ cells/mL.

Using enriched media VM2 which contains 10 mM of glutamine compared to 5 mM in standard R3 media, the glutamine concentrations could be kept well above 2 mM even at CSPR rates as low as 5.5 vol/day. No impact on growth was observed under these conditions.

While the present teachings are described in conjunction with various embodiments, it is not intended that the present teachings be limited to such embodiments. On the contrary, the present teachings encompass various alternatives, modifications, and equivalents, as will be appreciated by those of skill in the art. Furthermore, all literature and similar material cited in this application, including but not limited to, patents, patent applications, articles, books, treatises, are expressly incorporated herein by reference in their entirety for any purpose. The section headings used herein are for organizational purposes only and are not to be construed as limiting the subject matter described in any way.

We claim:

1. A perfusion bioreactor culture system, comprising:
a bioreactor configured to contain a tissue culture fluid and cells to be cultured;
a cell retention device configured to receive tissue culture fluid containing cells from the bioreactor, separate some cells from the tissue culture fluid and provide harvest output of tissue culture fluid and cells, and provide a recirculation output of tissue culture fluid and cells to the bioreactor;
wherein the system has a starting perfusion rate, a starting bioreactor volume, a starting cell retention device volume, and a starting volume ratio of the starting bioreactor volume and the starting cell retention device volume;
wherein either the starting perfusion rate is decreased, resulting in increased residence time of the cells in the bioreactor and the cell retention device, or
the starting bioreactor volume is increased or the starting cell retention volume is decreased, or both, resulting in an increase in the starting volume ratio.

2. The perfusion bioreactor culture system of claim 1, wherein the starting perfusion rate is decreased, resulting in increased residence time of the cells in the bioreactor and the cell retention device, and the starting bioreactor volume is increased or the starting cell retention volume is decreased, or both, resulting in an increase in the starting volume ratio.

3. The perfusion bioreactor culture system of claim 2, wherein the increase in the starting volume ratio is about the same proportion as the decrease in the starting perfusion rate.

4. The perfusion bioreactor culture system of claim 2, wherein the starting perfusion rate is decreased by up to about a third.

5. The perfusion bioreactor culture system of claim 2, wherein the starting perfusion rate is decreased by up to about a half.

6. The perfusion bioreactor culture system of claim 2, wherein the starting bioreactor volume is increased by about a third.

7. The perfusion bioreactor culture system of claim 2, wherein the starting bioreactor volume is increased by up to about a half.

8. The perfusion bioreactor culture system of claim 2, wherein the starting cell retention volume is decreased by up to about a third.

9. The perfusion bioreactor culture system of claim 2, wherein the starting cell retention volume is decreased by up to about a half.

10. The perfusion bioreactor culture system of claim 2, wherein the cells are mammalian cells.

11. The perfusion bioreactor culture system of claim 10, wherein the mammalian cells are selected from the group consisting of BHK cells, CHO cells, HKB cells, HEK cells, and NS0 cells.

12. The perfusion bioreactor culture system of claim 11, wherein the mammalian cells are BHK cells.

13. The perfusion bioreactor culture system of claim 10, wherein the mammalian cells are recombinant cells expressing recombinant factor VIII (rHFVIII).

14. The perfusion bioreactor culture system of claim 13, wherein the rHFVIII is an active ingredient of KG-FS.

15. The perfusion bioreactor culture system of claim 2, wherein the starting perfusion rate is about 1 to 15 volumes per day.

16. The perfusion bioreactor culture system of claim 2, wherein the increase in the starting volume ratio is up to about a third.

17. The perfusion bioreactor culture system of claim 2, wherein the increase in the starting volume ratio is up to about a half.

18. A method of optimizing a perfusion bioreactor system, comprising:
providing tissue culture fluid containing cells to a bioreactor system comprising a bioreactor and a cell retention device, wherein the system has a starting perfusion rate, a starting bioreactor volume, a starting cell retention device volume, and a starting volume ratio of the starting bioreactor volume and the starting cell retention volume; and
either decreasing the starting perfusion rate, resulting in increased residence time of the cells in the bioreactor and the cell retention device, or
increasing the starting bioreactor volume or decreasing the starting cell retention device volume, or both, resulting in an increase in the starting volume ratio.

19. The method of claim 18, further comprising:
decreasing the starting perfusion rate, resulting in increased residence time of the cells in the bioreactor and the cell retention device, and
increasing the starting bioreactor volume or decreasing the starting cell retention device volume, or both, resulting in an increase in the starting volume ratio.

20. The method of claim 18, wherein the increase in the starting volume ratio is in about a same proportion as the decrease in the starting perfusion rate.

21. The method of claim 18, wherein the starting perfusion rate is decreased by up to about a third.

22. The method of claim 18, wherein the starting perfusion rate is decreased by up to about a half.

23. The method of claim 18, wherein the starting bioreactor volume is increased by up to about a third.

24. The method of claim 18, wherein the starting bioreactor volume is increased by up to about half.

25. The method of claim 18, wherein the starting cell retention volume is decreased by up to about a third.

26. The method of claim 18, wherein the starting cell retention volume is decreased by up to about a half.

27. The method of claim 18, wherein the cells are mammalian cells.

28. The method of claim 27, wherein the mammalian cells are selected from the group consisting of BHK cells, CHO cells, HKB cells, HEK cells, and NS0 cells.

29. The method of claim 27, wherein the mammalian cells are BHK cells.

30. The method of claim 26, wherein the mammalian cells are recombinant cells expressing recombinant human factor VIII (rhFVIII).

31. The method of claim 29, wherein the rHFVIII is an active ingredient of KG-FS.

32. The method of claim 18, wherein the starting perfusion rate is about 1 to 15 volumes per day.

33. The method of claim 18, wherein the increase in the starting volume ratio is up to about a third.

34. The method of claim 18, wherein the increase in the starting volume ratio is up to about a half.

35. A method of optimizing a perfusion bioreactor system, comprising:
providing a first tissue culture fluid containing cells to a bioreactor system comprising a bioreactor and a cell retention device, wherein the system has a starting perfusion rate, a starting bioreactor volume, and a starting cell retention volume; and
decreasing the starting perfusion rate, resulting in increased residence time of the cells in the bioreactor and the cell retention device, and substituting the first tissue culture fluid for a second tissue culture fluid that has, compared to the first tissue culture fluid, increased concentrations of individual components of the first tissue culture fluid, without adding new components.

36. The method of claim 35, wherein the cells are mammalian cells.

37. The method of claim 35, wherein the mammalian cells are selected from the group consisting of BHK cells, CHO cells, HKB cells, HEK cells, and NS0 cells.

38. The method of claim 36, wherein the mammalian cells are BHK cells.

39. The method of claim 35, wherein the mammalian cells are recombinant cells expressing recombinant human factor VIII (rhFVIII).

40. The method of claim 39, wherein the rHFVIII is an active ingredient of KG-FS.

41. A method of optimizing a perfusion bioreactor system, comprising:
providing a first tissue culture fluid containing cells that express a recombinant protein to a bioreactor system comprising a bioreactor and a cell retention device, wherein the system has a starting perfusion rate, a starting bioreactor volume, and a starting cell retention device volume; and
decreasing the starting perfusion rate, resulting in increased residence time of the cells in the bioreactor and the cell retention device, and adding a stabilizer of the degradation of the recombinant protein.

42. The method of claim 41, wherein the cells are mammalian cells.

43. The method of claim 42, wherein the mammalian cells are selected from the group consisting of BHK cells, CHO cells, HKB cells, HEK cells, and NS0 cells.

44. The method of claim 42, wherein the mammalian cells are BHK cells.

45. The method of claim 42, wherein the mammalian cells are recombinant cells expressing factor VIII (rhFVIII).

46. The method of claim 45, wherein the rHFVIII is an active ingredient of KG-FS.

47. The method of claim 41, wherein the stabilizer is calcium.
